(19) United States
(12) Patent Application Publication
(10) Pub. No.: US 2022/0031970 A1
(43) Pub. Date: Feb. 3, 2022

(54) CAPSULE RETAINING SYSTEM

(71) Applicant: BN Intellectual Properties, Inc., St. Petersburg, FL (US)

(72) Inventors: Mark J. HOYT, Midvale, UT (US); Chad S FRAMPTON, American Fork, UT (US)

(21) Appl. No.: 16/945,284

(22) Filed: Jul. 31, 2020

(51) Int. Cl. A61M 15/00; A61M 11/00

(52) U.S. Cl. CPC A61M 15/003; A61M 11/00; A61M 2207/00; A61M 2205/0294; A61M 2205/0216

(57) ABSTRACT

A holding device including a sleeve wall having an outer wall surface and an inner wall surface. The inner wall surface forms a capsule insertion channel extending through an entirety of the sleeve wall. A lip is at an end of the capsule insertion channel and extends toward a center of the capsule insertion channel. The inner wall surface has a recess at a first distance along the inner wall surface from the lip, a base extending outward from the outer wall surface of the sleeve wall; and a flexible retaining element in the recess. The recess being configured to prevent movement of the flexible retaining element along the inner wall. The flexible retaining element having a cross-sectional profile with a first width at a side closer to the lip, and a second width at a side farther from the lip, the first width being larger than the second width.

### BACKGROUND

Conventional nebulizers are breathing machines used to treat lung conditions such as asthma, cystic fibrosis, and other respiratory illnesses. Nebulizers are used to administer medication in the form of a mist that is inhaled into the lungs. Nebulizers are often used in situations in which using a pressurized inhaler is difficult or ineffective, as well as to limit the side effects of medications like steroids by delivering the medicine directly to the respiratory system.

Atomizing type nebulizers, or “compressor nebulizers,” use an aerosol compressor to vaporize droplets of medicine. Ultrasonic type nebulizers, or “mesh nebulizers,” use high-frequency vibrations to make liquid medicine breathable. While ultrasonic models produce results comparable to jet nebulizers, they offer faster delivery of medication and operate more quietly.

Nebulizers come in tabletop or portable models. Tabletop nebulizers are intended for home or other fixed location use, as they need to be plugged into an electrical outlet. In some instances, portable units are battery-powered and small enough to fit into a bag or purse for travel and able to be transported by a single person often in one hand. Portable nebulizers tend to be more expensive and require disposable or rechargeable batteries or a power adapter to operate.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a sectional view of a capsule retaining system in accordance with some embodiments.

FIG. 2 is an enlarged sectional view of the capsule retaining system of FIG. 1 in accordance with some embodiments.

FIG. 3 is a sectional view of a capsule retaining system before inserting a capsule in accordance with some embodiments.

FIG. 4 is a sectional view of a capsule retaining system having a capsule partially inserted in accordance with some embodiments.

### DETAILED DESCRIPTION

The following disclosure provides many different embodiments, or examples, for implementing different features of the provided subject matter. Specific examples of components, values, operations, materials, arrangements, or the like, are described below to simplify the present disclosure. These are, of course, merely examples and are not intended to be limiting. Other components, values, operations, materials, arrangements, or the like, are contemplated. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

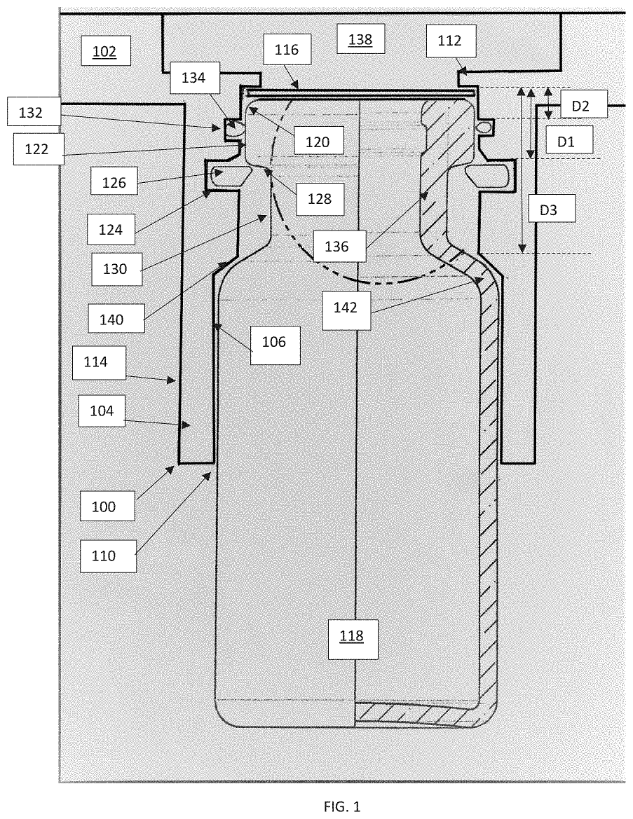

FIG. 1 is a sectional view of a capsule retaining system 100 in accordance with some embodiments.

In FIG. 1, capsule retaining device 100 includes, among other things, a base 102 and a sleeve wall 104. The sleeve wall 104 protrudes from base 102 in a direction perpendicular to the base 102. The sleeve wall 104 has an inner wall surface 106 extends from a distal end of the sleeve wall 104 to a bottom surface of a lip that is located at a top end of the sleeve wall 104. In some embodiments, the inner wall surface 106 is cylindrical shaped or a portion of the inner wall surface 106 is cylindrical shaped. The inner wall surface 106 forms an insertion channel 110 extending through an entirety of the sleeve wall 104. Insertion channel 110 extends from an open end of the sleeve wall 104 to the intersection of the sleeve wall 104 and base 102. In some embodiments, the insertion channel 110 is cylindrical-shaped. The lip 112 is formed at a top end of the insertion channel 110, and extends toward a center of the insertion channel 110. The lip 112 forms over a top end of the sleeve wall 104, and extends outward from an outer wall surface 114 of the sleeve wall 104. An annular seal 116 is positioned against the lip 112 at the top end of the insertion channel 110, i.e., the intersection of the insertion channel 110 and base 102. Examples of the annular seal 116 include a flat disk or ring of elastically-pliable material, e.g., silicone, rubber or similar materials. The annular seal 116 covers a top of a capsule 118 inserted in insertion channel 110 to provide a leak-resistant seal for the capsule 118 after insertion into capsule retaining device 100. The capsule 118 has a capsule neck upper end 120 on an upper capsule neck 122. The capsule 118 is configured to contain a liquid (not shown) which is converted into inhalable droplets by the nebulizer. The liquid exits the opening of the capsule, and passes through the opening in the annular seal 116 at the top of the capsule 118 to reach the droplet-producing portion of a nebulizer.

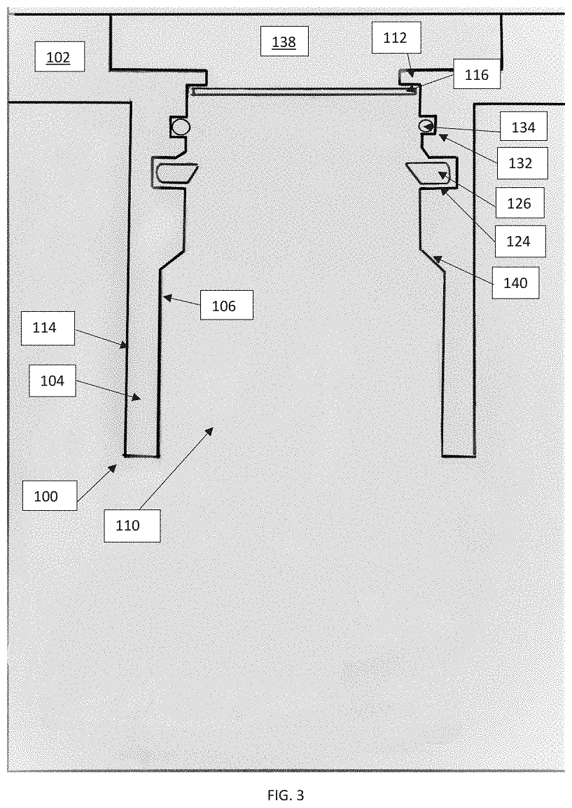

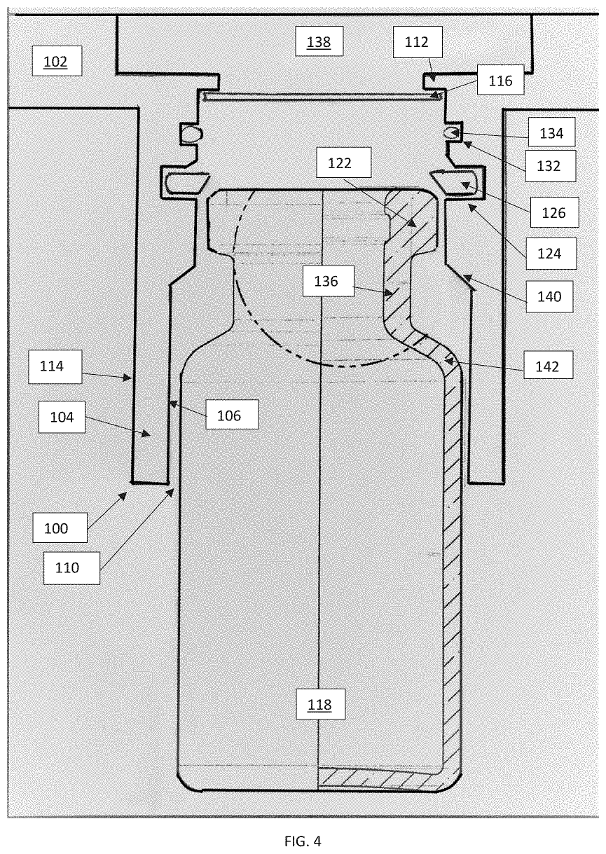

A first recess 124 is formed at a first distance D1 along the inner wall surface 106 from the lip 112. The first recess 124 has a depth extending into the inner wall surface 106 away from the center of the insertion channel 110. In some embodiments, the first recess 124 has a rectangular cross-section. A first flexible retaining element 126 is formed in the first recess 124. The first flexible retaining element 126 has a cross-sectional profile as seen in FIG. 1 with one end having an inner diameter greater than the other end. With the capsule 118 inserted into the insertion channel 110, an upper end 128 of a lower capsule neck 130 is engaged with the first flexible retaining element 126. FIG. 3 is a cross-sectional view of capsule retaining device 100 prior to insertion of capsule 118 into the insertion channel 110. In FIG. 3, the capsule 118 is not shown for clarity. FIG. 4 is a cross-sectional view of capsule 118 inserted into the insertion channel 110 prior to contacting flexible retaining element 126.

As shown in FIG. 1, the first flexible retaining element 126 is engaged with the lower capsule neck 130 to retain the capsule 118 in the insertion channel. The asymmetric cross-sectional profile of the first flexible retaining element 126 is configured to provide different degrees of resistance as the capsule 118 is inserted into the insertion channel 110. The first flexible retaining element 126 provides a low initial resistance when inserting the capsule 118, so that little force is needed when inserting the capsule 118. In contrast, the first flexible retaining element 126 provides a high initial resistance upon removal of the capsule 118 to prevent the capsule from being removed accidentally. In the given embodiment, the resistance imparted by first flexible retaining element 126 increases as the capsule is inserted into the insertion channel 110. In the given embodiment, after the upper end 128 of the capsule 118 is fully inserted in the insertion channel 110 past the first flexible retaining element 126, equal or greater resistance is provided by the first flexible retaining element 126 to retain capsule 118 in position as compared to the resistance imparted to prevent insertion of capsule 118 into the insertion channel 110. Examples of the second flexible retaining element include an O-ring or other fitting with an angled inner surface.

A second recess 132 is formed at a second distance D2 along the inner wall surface 106 from the lip 112. The second recess 132 has a depth d2 extending into the inner wall surface 106 away from the center of the insertion channel 110. In some embodiments, the depth of the second recess 132 is less than a depth d1 of the first recess 124. In some embodiments, the second recess 132 has a rectangular cross-section. The second recess 132 is located between the first recess 124 and the lip 112, and the second distance D2 is less than the first distance D1. A second flexible retaining element 134 is located in the second recess 132. Second flexible retaining element seals around the vial neck. In some embodiments, the second recess 132 can have a round cross-section or cross-section of other shapes that matches the shape of the second flexible retaining element 134. The second flexible retaining element 134 has a thickness in a direction different from the longitudinal direction of the sleeve wall 104. The thickness of the second flexible retaining element 134 is less than a thickness of the first flexible retaining element 126. The second flexible retaining element 134 has a constant inner diameter to retain the capsule 118 in the insertion channel 110. With the capsule 118 inserted into the insertion channel 110, the upper capsule neck portion 122 of the capsule neck 136 engages with the second flexible retaining element 134. The second flexible retaining element 134 is configured to retain the capsule 118 in a centered-position of the insertion channel 110 with respect to a piezoelectric grid recess 138 over the lip 112. Examples of the second flexible retaining element include an O-ring or other fitting element with a constant inner diameter.

A sleeve shoulder 140 is formed at a third distance D3 along the inner wall surface 106 from the lip 112. The third distance D3 is greater than the second distance D2. The sleeve shoulder 140 is configured to retain the capsule shoulder 142 in a centered-position of the insertion channel 110 with respect to the sleeve wall 104. In the meantime, the sleeve shoulder 140 will prevent the capsule 180 from over-insertion into the insertion channel 110 and damage the annular seal 116.

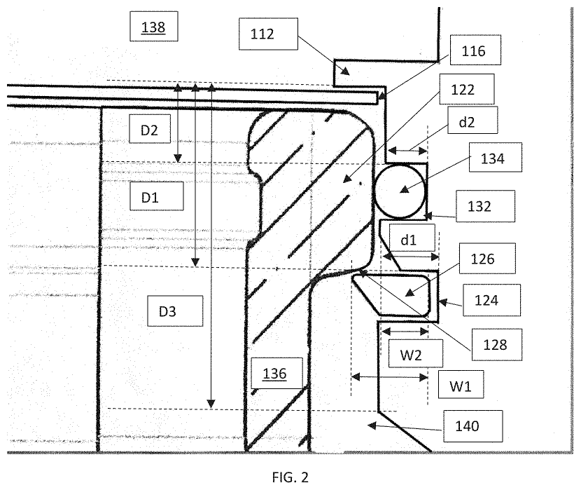

FIG. 2 is an enlarged sectional view of the capsule retaining system of FIG. 1 in accordance with some embodiments.

In FIG. 2, the first recess 124 is formed at a first distance D1 from the lip 112, and the second recess 132 is formed at a second distance D2 from the lip 112. The first distance D1 is greater than the second distance D2. The sleeve shoulder 140 is formed at a third distance D3 from the lip 112, and the third distance D3 is greater than the first distance D1 and the second distance D2.

The first retaining element 126 is located in the first recess 124. The first retaining element has a first width W1 at the upper side of the first retaining element 126 and a second width W2 at the lower side of the first retaining element 126. The first width W1 is greater than the second width W2. The upper side of the first retaining element 126, having the first width W1, is closer to the lip 112 than the lower side of the first retaining element 126, having the second width W2. An inner surface of the first retaining element is straight as the surface extends from the first width W1 to the second width W2.

When the position of the capsule neck 136 of the capsule 118 is secured in the insertion channel, the capsule neck lower end 128 is in contact with the upper side of the first retaining element 126. The center of the upper capsule neck 122 is in contact with an inner side of the second retaining element 134. The top of the capsule 118 is in contact with a bottom side of the annular seal 116, and a top side of the annular seal 116 is in contact with a bottom side of the lip 112.

In an embodiment, a holding device includes a sleeve wall having an outer wall surface and an inner wall surface, where the inner wall surface forms a capsule insertion channel extending through an entirety of the sleeve wall, and where a lip is at an end of the capsule insertion channel, the lip extending toward a center of the capsule insertion channel, the inner wall surface having a recess at a first distance along the inner wall surface from the lip. The holding device includes a base extending outward from the outer wall surface of the sleeve wall at a same end of the sleeve wall as the lip; an annular seal against the lip and within the capsule insertion channel; and a flexible retaining element in the recess, where, the recess is configured to prevent movement of the flexible retaining element along the inner wall with respect to the lip, and where the flexible retaining element has a cross-sectional profile with a first width at a side closer to the lip, and a second width at a side farther from the lip, where the first width is larger than the second width.

In an embodiment, the holding device where the inner wall surface further include a second recess at a second distance along the inner wall surface from the lip. The second distance is less than the first distance. The holding device further include a second flexible retaining element in the second recess. The second flexible retaining element has a constant inner diameter. The inner wall surface further include a sleeve shoulder at a third distance along the inner wall surface from the lip. The annular seal is a ring of elastically-pliable material. The base further include a piezoelectric grid recess over the lip.

In an embodiment, a capsule retaining device includes a sleeve wall having an inner wall surface forming an insertion channel extending through an entirety of the sleeve wall. The capsule retaining device includes a lip over a top of the insertion channel and extending toward a center of the insertion channel. The capsule retaining device includes a base over the lip and extending in a direction different from a longitudinal direction of the sleeve wall. The capsule retaining device includes an annular seal against the lip and within the insertion channel. The capsule retaining device includes where the inner wall surface further include a first recess and a second recess along the inner wall surface, and the second recess is between the lip and the first recess, and the inner wall surface further include a first flexible retaining element in the first recess and a second flexible retaining element in the second recess, where the second flexible retaining element has a thickness in a direction different from the longitudinal direction of the sleeve wall, the thickness of the second flexible retaining element is less than a thickness of the first flexible retaining element, and the first flexible retaining element has a first inner diameter of a top end less than a second inner diameter of a lower end.

In an embodiment, the capsule retaining device has the second flexible retaining element has a constant inner diameter. The annular seal is a ring of elastically-pliable material. The base further include a piezoelectric grid recess over the lip. The sleeve wall further include a sleeve shoulder at a third distance along the inner wall surface from the lip.

In an embodiment, a method of applying a capsule to a holding device includes providing the holding device having a capsule guiding sleeve, where the capsule guiding sleeve further include an insertion channel extending through an entirety of the capsule guiding sleeve. The method of applying includes aligning a capsule containing a liquid to an opening of the insertion channel. The method of applying includes inserting the capsule into the insertion channel. The method of applying includes retaining the capsule by engaging a lower capsule neck with a first flexible retaining element located in a first recess along an inner wall surface of the insertion channel, where the first flexible retaining element has an asymmetric cross-sectional profile to provide different degrees of resistance. The method of applying includes adjusting the capsule to a centered-position of the insertion channel by engaging an upper capsule neck with a second flexible retaining element, where the second flexible retaining element is located in a second recess along the inner wall surface of the insertion channel; and pressing a top end of the upper capsule neck against an annular seal, where the annular seal is against a lip at an end of the insertion channel.

In an embodiment, the method further includes where the holding device further include a base over the lip extending in a direction perpendicular to the capsule guiding sleeve. The base further include a piezoelectric grid recess over the lip. The second recess is between the lip and the first recess. An inner diameter of the first flexible retaining element varies in a direction aligned with the inner wall surface. The second flexible retaining element has an inner diameter slightly smaller than an outer diameter of the upper capsule neck. The annular seal is a ring of elastically-pliable material.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A holding device, comprising:
a sleeve wall having
an outer wall surface and
an inner wall surface, wherein the inner wall surface forms a capsule insertion channel extending through an entirety of the sleeve wall, and wherein a lip is at an end of the capsule insertion channel, the lip extending toward a center of the capsule insertion channel, the inner wall surface having a recess at a first distance along the inner wall surface from the lip;
a base extending outward from the outer wall surface of the sleeve wall at a same end of the sleeve wall as the lip;
an annular seal against the lip and within the capsule insertion channel; and
a flexible retaining element in the recess, wherein,
the recess is configured to prevent movement of the flexible retaining element along the inner wall with respect to the lip, and wherein
the flexible retaining element has a cross-sectional profile with a first width at a side closer to the lip, and a second width at a side farther from the lip, wherein the first width is larger than the second width.

2. The holding device of claim 1, wherein the inner wall surface further comprises a second recess at a second distance along the inner wall surface from the lip.

3. The holding device of claim 2, wherein the second distance is less than the first distance.

4. The holding device of claim 2, wherein the holding device further comprises a second flexible retaining element in the second recess.

5. The holding device of claim 4, wherein the second flexible retaining element has a constant inner diameter.

6. The holding device of claim 1, wherein the inner wall surface further comprises a sleeve shoulder at a third distance along the inner wall surface from the lip.

7. The holding device of claim 1, wherein the annular seal is a ring of elastically-pliable material.

8. The holding device of claim 1, wherein the base further comprises a piezoelectric grid recess over the lip.

9. A capsule retaining device, comprising:
a sleeve wall having an inner wall surface forming an insertion channel extending through an entirety of the sleeve wall;
a lip over a top of the insertion channel and extending toward a center of the insertion channel;
a base over the lip and extending in a direction different from a longitudinal direction of the sleeve wall; and
an annular seal against the lip and within the insertion channel,
wherein
the inner wall surface further comprises a first recess and a second recess along the inner wall surface, and the second recess is between the lip and the first recess, and
the inner wall surface further comprises a first flexible retaining element in the first recess and a second flexible retaining element in the second recess, wherein
the second flexible retaining element has a thickness in a direction different from the longitudinal direction of the sleeve wall, the thickness of the second flexible retaining element is less than a thickness of the first flexible retaining element, and
the first flexible retaining element has a first inner diameter of a top end less than a second inner diameter of a lower end.

10. The capsule retaining device of claim 9, wherein the second flexible retaining element has a constant inner diameter.

11. The capsule retaining device of claim 9, wherein the annular seal is a ring of elastically-pliable material.

12. The capsule retaining device of claim 9, wherein the base further comprises a piezoelectric grid recess over the lip.

13. The capsule retaining device of claim 9, wherein the sleeve wall further comprises a sleeve shoulder at a third distance along the inner wall surface from the lip.

14. A method of applying a capsule to a holding device, the method comprising:
providing the holding device having a capsule guiding sleeve, wherein the capsule guiding sleeve further comprises an insertion channel extending through an entirety of the capsule guiding sleeve;
aligning a capsule containing a liquid to an opening of the insertion channel;
inserting the capsule into the insertion channel;
retaining the capsule by engaging a lower capsule neck with a first flexible retaining element located in a first recess along an inner wall surface of the insertion channel, wherein the first flexible retaining element has an asymmetric cross-sectional profile to provide different degrees of resistance;
adjusting the capsule to a centered-position of the insertion channel by engaging an upper capsule neck with a second flexible retaining element, wherein the second flexible retaining element is located in a second recess along the inner wall surface of the insertion channel; and
pressing a top end of the upper capsule neck against an annular seal, wherein the annular seal is against a lip at an end of the insertion channel.

15. The method of claim 14, wherein the holding device further comprises a base over the lip extending in a direction perpendicular to the capsule guiding sleeve.

16. The method of claim 14, wherein the second recess is between the lip and the first recess.

17. The method of claim 14, wherein an inner diameter of the first flexible retaining element varies in a direction aligned with the inner wall surface.

18. The method of claim 14, wherein the second flexible retaining element has an inner diameter slightly smaller than an outer diameter of the upper capsule neck.

19. The method of claim 14, wherein the annular seal is a ring of elastically-pliable material.

20. The method of claim 15, wherein the base further comprises a piezoelectric grid recess over the lip.
